SEQUENCE LISTING (6 sequences)

SEQ ID NO: 1 - DNA, 35 bp, Artificial
  Designed polynucleotide for primer Primer for cloning CaMKII (delta) gene
ccacccaccaccaccaatggcttcgaccaccacct

SEQ ID NO: 2 - DNA, 20 bp, Artificial
  Designed polynucleotide for primer Primer for cloning CaMKII (delta) gene
tcagatgttttgccacaaag

SEQ ID NO: 3 - DNA, 37 bp, Artificial
  Designed polynucleotide for primer Primer for cloning of eIF4B gene
ccacccaccaccaccaatggcggcctcagcaaaaaag

SEQ ID NO: 4 - DNA, 22 bp, Artificial
  Designed polynucleotide for primer Primer for cloning of eIF4B gene
ctattcggcataatcttctccc

SEQ ID NO: 5 - DNA, 34 bp, Artificial
  Designed polynucleotide for primer Primer for cloning of Stress-induced phosphoprotein 1 gene
ccacccaccaccaccaatggagcaggtcaatgag

SEQ ID NO: 6 - DNA, 18 bp, Artificial
  Designed polynucleotide for primer Primer for cloning of Stress-induced phosphoprotein 1 gene
tcaccgaattgcaatcag

(19) United States
(12) Patent Application Publication
(10) Pub. No.: US 2007/0134734 A1
(43) Pub. Date: Jun. 14, 2007

(54) Novel screening method of indicator substance

(75) Inventors: Yaeta Endo, Ehime (JP); Tatsuya Sawasaki, Ehime (JP)

Correspondence Address: KILYK & BOWERSOX, P.L.L.C., 400 HOLIDAY COURT, SUITE 102, WARRENTON, VA, 20186, US

(73) Assignees: CellFree Sciences Co., LTD., Matsuyama-shi (JP); Tatsuya Sawasaki, Matsuyama-shi (JP)

(21) Appl. No.: 10/575,807

(22) PCT Filed: Oct. 14, 2004
(86) PCT No.: PCT/JP04/15122

§ 371 Date: Apr. 13, 2006

(30) Foreign Application Priority Data
Oct. 14, 2003 (JP) JP2003-353949

(51) Int. Cl. G01N 33/567; C07K 16/18

(52) U.S. Cl. 435/7.2; 530/388.25

(57) ABSTRACT

An object of the present invention is to provide a novel means for finding a passive substance to the action of a selected trigger protein in a target cell system. In particular, the present invention succeeded in establishing a novel method for screening an indicator substance by the steps of contacting the trigger protein with a target cell extract to initiate the action on an unspecified indicator substance by the trigger protein, and specifying the substance changed by the action induced by the trigger protein. The primary part of the present invention comprises the following steps: 1) contacting a trigger protein prepared by a cell-free protein synthesis means with a target cell extract which contains the indicator substance that is passively produced by an action induced by the trigger protein and desired to screen, to initiate the action by the trigger protein, and 2) specifying the substance changed by the action induced by the trigger protein.

### TECHNICAL FIELD

The present invention relates to a novel method for screening an indicator substance passively affected by an action induced by a trigger protein. In more detail, the present invention relates to a method for screening an indicator substance involved in an action induced by a trigger protein, comprising the following steps: contacting the trigger protein with a target cell extract, to initiate the action on an unspecified indicator substance by the trigger protein, and specifying a substance changed by the action induced by the trigger protein. Further, the present invention comprises using a cell-free protein synthesis means to carry out this screening method. Further, the present invention comprises a method for screening a substance affecting a trigger protein's action on a target cell extract using this system.

### BACKGROUND ART

Proteome analysis which analyzes proteins involved in an intracellular signaling system, in particular phosphorylated proteins is roughly divided into expression proteomics and functional (interaction) proteomics.

While the expression proteomics is an approach to comprehensively analyze where and how much a certain protein expresses in vivo, the functional proteomics is an approach to comprehensively analyze with what molecule the certain protein interacts. Traditionally, these analyses have been carried out by such method as immunoprecipitation, pull-down, Yeast 2-Hybrid, phage display and the like.

Immunoprecipitation utilizes a phenomenon wherein a soluble polymer antigen such as protein and polysaccharide reacts with an antiserum or antibody to produce an insoluble antigen-antibody complex, which is then precipitated. It is a method for using a specific antibody to precipitate a protein in interest from proteins in cells, analyzing the protein and other proteins simultaneously precipitated, and screening for a protein which can bind to the protein in interest.

Pull-down method is a method for analyzing the binding mainly between proteins themselves in vitro. Specifically, it is a screening method in which one protein is precipitated to screen for the other co-precipitated protein.

Yeast 2-Hybrid method is a method for using a fusion protein of a DNA-binding domain with a target and fusing the other protein fragment to an activating domain to confirm which interacts with the target to reconstruct an active transcription factor and induces a reporter gene's action.

Phage display method is a screening method for expressing random fusion proteins on the surface of phage particles to screen for those among these random fusion proteins which interact with the target substance.

The methods indicated above are suitable for screening for only one protein which interacts with a particular protein, but can not be applied to a screening for one or more of complex proteins involved in a plurality of signaling systems. In addition, they can not be applied to a screening which targets an intermediate protein involved in a plurality of signaling systems or a protein which is transiently phosphorylated.

Therefore, the conventional screening methods can identify no element but the products or end products from the early reaction of a series of phosphorylation, which are parts of elements in the intracellular signaling pathway.

However, intermediates and complex proteins by a series of intracellular phosphorylation are believed to be important targets to identify for researches in pharmacology and toxicology and research for possible endocrine disrupting compounds, and hence a method for screening proteins involved in a plurality of these intracellular signaling systems cells is desired to construct.

On the other hand, in order to obtain efficiently various proteins required for screening researches described above, attention has been attracted to a cell-free protein synthesizing means today. In this method, rabbit reticulocyte cell-free system (Reticulocyte Lysate) has often been used. However, recently, based on the elucidation of destabilization mechanism of a wheat embryo cell-free system (Wheat Embryo Extract), a method for preparing a wheat embryo extract solution having stability and a high translation activity, and a highly efficient cell-free protein synthesis system using the wheat embryo extract solution have been provided and applied in various protein synthesis. (nonpatent document No. 1) (patent document Nos. from 1 to 3). Further, researches on reagents for fabricating protein chips appropriate to screening for multiple analyses using a wheat embryo cell-free system have been conducted.

Patent Document No. 1: Japanese Patent Application Laid-open No. 2000-236896

Patent Document No. 2: Japanese Patent Application Laid-open No. 2002-125693

Patent Document No. 3: Japanese Patent Application Laid-open No. 2002-204689

Nonpatent Document No. 1: Proc. Natl. Acad. Sci. USA, 99: 14652-14657 (2002)

### DISCLOSURE OF THE INVENTION

An object of the present invention is to provide a novel means to find a passive substance to the action of the selected trigger protein in target cell system.

We have strenuously studied to solve the matters described above and finally succeeded in establishing novel methods for screening an indicator substance by the steps of contacting a trigger protein with a target cell extract, initiating an action by the trigger protein on an unspecified indicator substance, and specifying the substance changed by an action induced by the trigger protein. Further, it is confirmed that the in vitro screening method using a target cell extract clarifies the in vivo behavior of an indicator substance changed by an action induced by a trigger protein in a certain target cell system. As the facts above, the present invention is accomplished on the basis of these findings.

Therefore, the present invention consists of the followings:

1. A method for screening an indicator substance passively produced by an action induced by a trigger protein, comprising the following steps:

1) contacting the trigger protein prepared by a cell-free protein synthesizing means with a target cell extract which contains the indicator substance that is passively produced by an action induced by the trigger protein and desired to screen, to initiate the action by the trigger protein, and

2) specifying the substance changed by the action induced by the trigger protein.

2. The screening method according to the preceding 1, wherein the cell-free protein synthesizing means uses a wheat embryo extract which is substantially removed from a contaminating endosperm component and a low molecular protein synthesis inhibitory substance.

3. The screening method according to the preceding 2, wherein the unpurified or partially purified trigger protein prepared by the cell-free protein synthesizing means initiates the action on an unspecified indicator substance.

4. The screening method according to any one of the preceding 1 to 3, wherein as a marker for identifying an indicator substance changed by an action induced by a trigger protein, a particular substance capable of labeling the indicator substance of interest is introduced into the system.

5. The screening method according to the preceding 4, wherein the indicator substance changed by an action induced by a trigger protein is labeled with a substance selected from the followings:

1) radioactive substance,

2) fluorescent substance,

3) stable isotope, and

4) antibody.

6. The screening method according to any one of the preceding 1 to 3, wherein the indicator substance changed by the action induced by the trigger protein is detected using a change in molecular weight as a marker.

7. The screening method according to any one of the preceding 1 to 6, wherein the trigger protein is selected from the followings:

1) enzyme,

2) transcription factor,

3) intranuclear receptor, and

4) cell membrane receptor.

8. The screening method according to any one of the preceding 1 to 7, wherein the target cell extract is selected from the followings:

1) normal cell-derived extract,

2) cancer cell-derived extract,

3) wheat embryo extract, and

4) cell-derived extract subjected to stress and/or chemical treatment.

9. A reagent kit for screening, comprising at least one reagent used in the screening method according to any one of the preceding 1 to 8.

10. A novel indicator substance which is passively produced by an action induced by a trigger protein identified by the screening method according to any one of the preceding 1 to 8.

11. A method for screening a substance affecting the action of a trigger protein on a target cell extract, comprising: using the indicator substance specified in the preceding 10 as a control, contacting the trigger protein with the target cell extract in the presence or absence of the candidate substance, and comparing changes in the specified indicator substance.

### EFFECTS OF THE INVENTION

Novel methods for screening the indicator substance of the present invention are useful for discovering a novel regulatory system in vivo. This system permits a screening for a novel regulatory substance in an organism affecting system.

### DESCRIPTION OF THE PREFERRED EMBODIMENT

The present invention is a method for screening an indicator substance passively produced by an action induced by a trigger protein and comprises at least following steps.

Herein, trigger protein means a substance which can affect a bioactive system in a driving manner, covering many enzymes, transcription factors, receptor and the like. For example, kinase used as an enzyme is a trigger protein for phosphorylation, and an intranuclear receptor is a trigger protein for interaction between the nuclear receptor and its ligand.

In step 1, which is an essential step of the present invention, a selected trigger protein is contacted with a cell extract (target cell extract) which will be a target of the trigger, to initiate the action on an unspecified indicator substance (referred to as unspecified because a passive substance is still unclear at this time point) by the trigger protein.

Herein, contacting means broadly a state which allows an endogenous substance to give an action on other endogenous substances, usually, in a physiological solution. For example, kinase is allowed to phosphorylate its substrate in a target cell extract, and an intranuclear receptor is allowed to interact with its ligand. The target cell extract means a cell extract comprising an indicator substance passively produced by an action induced by a selected trigger protein, and the indicator substance is specified from among substances derived from this cell. The target cell extract used in the present invention can be obtained from normal cells, cancer cells, virus infected cells, cells derived from patients with inherited diseases, cells derived from patients with allergic disorders, and cells derived from patients with lifestyle-related diseases such as hypertension and diabetes, and the like. It is believed that their target cell extracts can be used to provide important information on a series of signaling systems, which is to elucidate the causes of cancers, virus infections and genetic diseases. Further, the target cell extract includes an extract from a wheat embryo, E. Coli or rabbit reticulocyte for use in a cell-free protein synthesis system. In addition, the target cell extract can be used to screen a substance which inhibits and/or facilitates a cell-free protein synthesis. Further, the extract includes extracts from the above described cells subjected to stress and/or chemical treatment. Herein, the stress treatment means to expose a cell, which is expected to provide an extract solution, to a stress such as low or high temperature, hypoxia, dryness, nutrient depletion, radiation, or virus infection in advance. Meanwhile, chemical treatment means to administer a cell, which is expected to provide an extract solution, with a biologically active substance such as hormones, cell growth factors, neurotransmitters, cytokines, autacoids, carcinogens, antibiotics, anticancer agents, hypotensive agents, antiviral agents, or pesticides in advance.

Such a cell extract can be obtained by centrifugation of a homogenized cell in a buffer by a conventional method. Conditions for extraction and centrifugation can be changed to permit collection of an extract from an organelle such as nucleus, mitochondria, Golgi apparatus, and endoplasmic reticulum, besides an extract derived from cytoplasm. Moreover, the trigger protein which has been contacted with a target cell extract initiates an action to allow a series of direct and/or indirect reactions, resulting in some kind of changes in an unspecified indicator substance. The unspecified indicator substance may either be known or unknown previously. Changes can easily be determined by comparison with the control system without the trigger protein acting.

Step 2, which is an essential step of the present invention, comprises a step for specifying a substance changed by an action induced by this trigger protein. This changed substance is specified as an indicator substance. If desired, in order to use widely in the field of protein chemistry, the specified substance is usually isolated by electrophoresis or column chromatography using a label or a particular indicator bound to its changed site, and identified by determining electrophoresis level, amino acid sequence, molecular weight, bioactivity, charge, affinity and the like. The novel systems as described above will provide a novel method for screening an indicator substance for the selected trigger protein.

An indicator substance changed by an action induced by a trigger protein needs to take any identifiable form. In a convenient method, a particular substance capable of labeling the indicator substance of interest can be introduced into the system as a marker for identification. For example, if kinase is used as a trigger protein and ATP which has been labeled with radioactive isotope ³²P is introduced into the system as its substrate, the phosphorylation activity of the trigger protein can label an indicator substance with this ³²P to trace the changes caused by the action of interest. In the present invention, a particular substance which can label an indicator substance of interest refers to such substance as this labeling ATP.

The means to label a substance changed by an action induced by this trigger protein may include any known means to apply, such as 1) radioactive substances, 2) fluorescent substances, 3) stable isotopes, and 4) antibodies. The means to identify a labeled indicator substance may include a known means which is optimized for the labeling means and is not limited in particular.

Further, the indicator for detecting a substance changed by an action induced by a trigger protein may be a change in molecular weight, though it is different from the change as described above. The indicator substance, which is previously identified or known, makes the detection easier.

Further, in addition to essential steps described above, after a trigger protein is expressed in a target cell with the gene encoding the protein introduced, the behavior of an indicator substance changed by the trigger protein and/or the action induced by the trigger protein is compared with the behavior indicated by the screening method of the present invention. The comparison can confirm whether the in vitro screening method using the target cell extract of the present invention represents the in vivo behavior of the indicator substance changed by the action by a trigger protein in a certain target cell system.

The general steps to the identification of an indicator substance are as follows.

1) A selected trigger protein is prepared.

2) A target cell extract to ask for screening an indicator substance produced by an action induced by the trigger protein is prepared.

3) The trigger protein is contacted with the target cell extract.

4) If desired, a particular substance which can label to the indicator substance is introduced into a system.

5) An action is initiated by the trigger protein.

6) The desired fraction treatment is conducted to specify a substance changed by the action induced by the trigger protein. For example, one-dimensional or two-dimensional electrophoresis is conducted.

7) The indicator substance is specified by the label. Specification is carried out by, for example, comparing them with the substance subjected to the above treatment in the absence of the trigger protein.

8) The specified indicator substance is isolated and identified to confirm a substance affected by the trigger protein in the target cell.

9) In the system described above, a candidate compound is added simultaneously with the addition of the trigger protein, allowing screening for a substance affecting the trigger protein's action on the target cell extract.

The screening system of the present invention is suitable for analysis of intracellular signaling, but not limited to this in particular. As preferred examples of trigger proteins applicable to the system of the present invention, 1) enzymes, 2) transcription factors and 3) intranuclear receptors may be mentioned. The enzyme includes preferably, but is not limited to, kinase, phosphatase, and protease. The transcription factor includes preferably a larger number of factors such as c-Fos and NF-1 which are reported and broadly applicable. The intranuclear receptor includes estrogen receptor, glucocorticoid receptor, or the other. Further, the cell membrane receptor includes EGFR (Epidermal growth factor receptor) and c-Met (hepatocyte growth factor receptor), or the other.

If kinase is used for a trigger protein, a protein involved in a transduction mechanism using phosphorylation as a signal can be specified. Phosphorylation is a universal posttranslational modification controlling many important cellular processes such as metabolism, proliferation/growth and differentiation. It is known that phosphorylation on a protein may cause various effects such as activating/inactivating the enzymatic activity of the protein or changing the binding affinity between certain proteins.

When phosphorylation occurs, a high energy of phosphoryl group is transferred from an adenosine triphosphate molecule (ATP) to a particular protein via a protein kinase. The protein kinases can be roughly classified based on amino acids to which a phosphoryl group is transferred. For example, a protein tyrosine kinase and a protein serine/threonine kinase specifically catalyze phosphorylations on tyrosine and serine or threonine residues respectively. Further, phosphorylation may be occurred in response to cell signals from hormones, neurotransmitters, proliferation/growth factor, differential factor, and the other molecules. A phosphoryl group can be enzymatically removed from a phosphorylated protein by a protein phosphatase. Moreover, kinase and phosphatase have an important action to activate many substrate molecules in a monomolecular manner, thereby to amplify intracellular signals through the action.

It is known that a protein kinase plays a key role in cell regulation, and it is reported that defects in the protein kinase are involved in many diseases and disorders, for example, signaling by inappropriately regulated protein kinases is involved in various diseases such as inflammation, cancer, arteriosclerosis and psoriasis. Further, the overexpression of cellular tyrosine kinases in epidermal growth factor receptor (EGFR) or platelet-derived growth factor receptor (PDGFR) or the mutation of tyrosine kinases which will constitutively produce active ones can be found in many cancer cells. (Nat. Med. 2: 561-66, (1996)). In addition, protein kinases are involved in inflammation signals. Defective Ser/Thr kinase genes are involved in a number of diseases such as myotonic dystrophy, cancer, and Alzheimer's disease.

For protein kinases described above, the following six kinds have been identified: (1) phosphorylase kinase, (2) myosin light-chain kinase, (3) phospholipid dependent protein kinase, (4) calmodulin kinase I, (5) calmodulin kinase II and (6) calmodulin kinase III. Of the six kinds, (5) calmodulin kinase II (CaMKII) has an autophosphorylation site at calmodulin (CaM) binding site, and Ca and CaM dependencies disappear when Thr286 (a) is phosphorylated. Further, the phosphorylation reaction of Thr305 (a) leads to the disappearance of CaM binding capacity. Therefore, autophosphorylation reaction is considered to be an activation reaction of an enzyme. CaMKII has broad substrate specificity and is engaged in various Ca dependent cell functions. CaMKII is one of protein kinases on the characters of which extensive studies have been carried out.

The system of an indicator substance specified in the present invention is effective in screening a novel disorder suppressing factors (substances). A target cell extract, for which an indicator substance is specified, is used to contact a trigger protein with the target cell extract in the presence and absence of a candidate substance and compare changes in the specified indicator substance, allowing screening a substance which affects the trigger protein's action on the target cell extract.

In addition, the in vitro screening method using the target cell extract of the present invention illustrates the in vivo behavior of an indicator substance changed by an action induced by a trigger protein in a certain target cell system.

One of the fundamental features of the present invention is that the trigger protein used in the screening system of the present invention can be easily synthesized with a cell-free protein synthesis system. As a cell-free protein synthesis system, a system using a wheat embryo extract in particular is the most appropriate. With the use of these systems, a variety of trigger proteins can be simultaneously synthesized in a large amount, thereby a high-throughput screening system may be constructed.

The trigger protein used in the present invention may be a protein purified from organism materials such as animals, plants, microorganisms and transgenic cells by a well known method or a protein artificially synthesized. The trigger proteins prepared by a method using a cell-free protein synthesis system as shown below can preferably be used. In addition, the trigger protein prepared with a cell-free protein synthesis system contains little contaminants. Therefore, the extract solution containing the trigger protein can be used as it is or as it is partially purified to initiate the action on an unspecified indicator substance in the target cell extract. This provides a great usefulness to the automation and acceleration of screening system. Herein, the term as it is partially purified means that it is freed from insoluble substances by a way such as centrifugation.

(1) Preparation of Cell-Free Protein Synthesis System

A cell-free protein synthesis system used in the present invention is a method wherein intracellularly existing components such as ribosome of protein translator apparatus are extracted from an organism to give an extract solution, to which a transcription or translation template, nucleic acids of a substrate, amino acids, energy source, various ions, buffer, and the other effective factors are then added to synthesize in vitro. The method includes the following types: the one using RNA as template (hereinafter sometimes referred to as “cell-free translation system”), and the one using DNA and further adding an enzyme such as RNA polymerase necessary for transcription to carry out the reaction (hereinafter sometimes referred to as “cell-free transcription/translation system.”). A cell-free protein synthesis system in the present invention includes both the cell-free translation system and the cell-free transcription/translation system described above.

For a cell-free protein synthesis system used in the present invention, a prepared material from microorganisms such as E. Coli, embryos of plant seeds, mammalian reticulocytes such as rabbit's and the like are used. For a cell-free protein synthesis system, a commercially available one can be used, and also it can be prepared from, for example, microorganisms described above, embryos, reticulocytes using a well known method per se, for example, it can be prepared from a cell extract-containing solution from a microorganism such as E. Coli using such a method as described in Pratt, J. M. et al., Transcription and Translation, Hames, 179-209, B. D. & Higgins, S. J., eds, IRL Press, Oxford (1984).

As commercially available cell-free protein synthesis system, the ones derived from E. Coli such as E. Coli S30 extract system (supplied by Promega) and RTS 500 Rapid Translation System (supplied by Roche), and the ones derived from rabbit reticulocytes such as Rabbit Reticulocyte Lysate System (supplied by Promega) may be mentioned.

However, for a cell-free protein synthesis system used in the present invention, gramineous plants such as wheat, barley, rice and corn are preferred. A cell-free protein synthesis system derived from a wheat embryo is particularly preferred.

A solution comprising a wheat embryo extract used in the present invention is commercially available as PROTEIOS™ (supplied by TOYOBO).

As a method for preparing a wheat embryo extract solution, for an isolation method of a wheat embryo, for example, such a method as described in Johnston, F. B. et al., Nature, 179, 160-161 (1957) may be used, while for an extraction method of a wheat embryo extract-containing solution from an isolated embryo, for example, such a method as described Erickson, A. H. et al., (1996) Meth. In Enzymol., 96, 38-50 can be used. In addition, International Publication WO 03/064671 may be mentioned.

The wheat embryo extract suitable for use in the present invention will be purified to be almost free from an endosperm containing substances (the substances such as tritin, thionine, and ribonuclease which act on mRNAs, tRNAs, protein translation factors, ribosome and others to inhibit those functions) which inhibit protein synthesis function that cells as materials have or keep in themselves. Herein, the wheat embryo extract purified to be almost free from an endosperm means a wheat embryo extract removed from an endosperm portion to such an extent that substantially no ribosome may be deadenylated. Such an extent that substantially no ribosome may be deadenylated means that a rate of less than 7% and preferably 1% or below of ribosome is deadenylated.

The wheat embryo extract described above may comprise a protein derived from a wheat embryo extract-containing solution (and independently supplied if necessary). In view of the preservation stability in a freeze-dried state, its usability and the like, the composition before freeze-dried comprises the protein preferably at a content of 1 to 10 weight %, and more preferably 2.5 to 5 weight % of the total composition, while the composition after freeze-dried comprises preferably the protein at a content of 10 to 90 weight %, and more preferably 25 to 70 weight % of the total lyophilized composition. But the content is not particularly limited. Now, the protein content herein can be calculated by determining its absorbance (260, 280 and 320 nm).

(2) Reduction of Deliquescent Substances in Wheat Embryo Extract-Containing Solution

The wheat embryo extract-containing solution described above contains extraction solvents or deliquescent substances such as potassium acetate and magnesium acetate originating from buffers used in gelfiltration after extraction. Consequently, the wheat embryo extract-containing solution has a problem that the solution is directly used to prepare a translation reaction solution, which is then lyophilized followed by dissolving again, resulting in deterioration in quality of the formulation. Deterioration in quality means that the formulation may not be completely dissolved in added water, thereby to decrease synthesis activities in protein synthesis reaction. Thus, the deliquescent substances may be reduced in concentration in the wheat embryo extract-containing solution to give no influence on the quality of the lyophilized formulation. The specific methods for reducing deliquescent substances include, for example, a gelfiltration method using a gel support equilibrated beforehand with a deliquescent substance-reduced or free solution, and a dialysis method. The deliquescent substances are reduced by using these methods to provide a solution for translation reaction which has a final deliquescent substance concentration of 60 mM or below. Specifically, the solution finally prepared for translation reaction should contain potassium acetate at a decreased concentration of 60 mM or below and preferably 50 mM or below. Furthermore, the lyophilized formulation has preferably a deliquescent substance content of 0.01 part by weight or below and particularly preferably 0.005 part by weight or below relative to 1 part by weight of the protein contained in the formulation to exempt itself from deterioration in the preservation stability of a freeze-dried state.

(3) Elimination of Contaminant Microbes

As the wheat embryo extract-containing solution can be contaminated with spores of microbes, in particular a filamentous bacterium (mold) and the like, these microbes are preferred to be eliminated. Microbial proliferation may be seen in particular in a long-term (a day or higher) cell-free protein synthesis reaction, and hence is important to inhibit. The means of eliminating microbes preferably includes, but is not limited to, a filter for filter sterilization to use. The filter may not be limited in pore size to a particular one in so far as it can eliminate contamination-suspected microbes, but has usually a pore size of 0.1 to 1 μm, and preferably 0.2 to 0.5 μm.

(4) Method to Remove Low Molecular Synthesis Inhibitors from Wheat Embryo Extract-Containing Solution

In addition to the foregoing operations, a step for removing low molecular synthesis inhibitors can be added anywhere in the preparation of the wheat embryo extract-containing solution to make the solution suitable for cell-free protein synthesis of a trigger protein with more preferable effects.

A wheat embryo extract-containing solution prepared by substantially removing an endosperm component contains low molecular synthesis inhibitors having a protein synthesis inhibitory activity (this may be referred to as “low molecular synthesis inhibitor”). Thus, the removal of them may provide a wheat embryo extract-containing solution having a high protein synthesis activity. Specifically, the removal is conducted by fractionally removing low molecular synthesis inhibitors from the components of a wheat embryo extract-containing solution through the differences in their molecular weights. The low molecular synthesis inhibitor can be fractionally removed to have a smaller molecular weight than the least factor among those factors necessary for protein synthesis that are contained in the wheat embryo extract-containing solution. Specifically, the inhibitor may be fractionally removed to have a molecular weight of 50,000 to 14,000 or less, or preferably of less than 14,000. As the method for removing low molecular synthesis inhibitors from a wheat embryo extract-containing solution, a method already known per se, for example, dialysis using a dialysis membrane, gelfiltration, or ultrafiltration can be used. Among them, dialysis is preferred in view of, for example, the easiness of supplying an internal solution for dialysis with materials.

As a dialysis membrane for use in the removing operation of low molecular synthesis inhibitors through dialysis, the one which can remove a molecule having a molecular weight of 50,000 to 12,000 may be mentioned, specifically, a recyclable cellulose membrane (from Viskase Sales, Chicago) which can remove a molecule having a molecular weight of 12,000 to 14,000, Spectra/Pore 6 (from SPECTRUM LABORATORIES INC., CA, USA) which can remove a molecule having a molecular weight of 50,000, and the like may preferably be used. A suitable amount of the wheat embryo extract-containing solution will be put toward the one-side of such dialysis membrane, and then dialysis is conducted by a conventional method. The dialysis is preferred to be conducted for 30 minutes to 24 hours.

While removing low molecular synthesis inhibitors, if an insoluble substance is produced in a wheat embryo extract-containing solution, inhibiting this production (hereinafter, this may be referred to as “the stabilization of a wheat embryo extract-containing solution”) allows the wheat embryo extract-containing solution finally prepared or a solution for translation reaction to have a higher protein synthesis activity. As a specific method of stabilizing a wheat embryo extract-containing solution or a solution for translation reaction, there is mentioned a method wherein low molecular synthesis inhibitors described above are removed from a wheat embryo extract-containing solution or a solution for translation reaction with a high energy phosphate compound such as ATP or GTP (hereinafter, they may be referred to as “stabilization component”) contained. As the high energy phosphate compound, ATP may preferably be used. Further, the removal may be preferably carried out from the solution with ATP and GTP, and more preferably ATP, GTP, and 20 kinds of amino acids contained.

The solution may be supplied with these stabilization components, incubated, and then subjected to the process for removing low molecular synthesis inhibitors. Alternatively, the stabilization component may be added also to an external solution for dialysis, and then the solution is subjected to dialysis to remove low molecular synthesis inhibitor. Advantageously, the stabilization component in the external solution for dialysis, even if decomposed during dialysis, can be constantly supplemented with the fresh stabilization component. This approach can be applied to gelfiltration and ultrafiltration used to give the same effect. The supports they use are equilibrated with a filtration buffer with a stabilization component contained, supplied with a wheat embryo extract-containing solution or a solution for translation reaction with a stabilization component contained, and supplied with the above-described buffer to filtrate.

The amount of a stabilization component to add and the time of stabilization to treat may be selected as appropriate depending on the kind of a wheat embryo extract-containing solution and the method of preparation. As the selection method, there may be mentioned a method wherein a wheat embryo extract-containing solution is supplied with various stabilization components in amount and kind on a trial basis, and subjected to the step for removing low molecular synthesis inhibitors after an appropriate hour to give a treated wheat embryo extract-containing solution, which is then centrifuged to separate into the soluble compartment and the insoluble component, thereby to select a case resulting in a less amount of the insoluble component. Alternatively, a method is preferred wherein the treated wheat embryo extract-containing solution is used to carry out cell-free protein synthesis, thereby to select a case resulting in a high protein synthesis activity. Further, a method is mentioned wherein appropriate stabilization component is also added in an external solution for dialysis, and then wheat embryo extract-containing solution is subjected to dialysis for an appropriate time, thereby to select based of the amount of the insoluble component in the solution thus obtained or the protein synthesis activity of the solution thus obtained.

As one example of the stabilizing condition of a wheat embryo extract-containing solution thus selected, specifically, if dialysis is carried out for the step of removing low molecular synthesis inhibitors, there is mentioned a method wherein the wheat embryo extract-containing solution and the external solution for dialysis are supplied with 100 μM to 0.5 mM of ATP, 25 μM to 1 mM of GTP and 25 μM to 5 mM of each 20 kinds of amino acids and then subjected to dialysis for 30 minutes to an hour or more. The temperature for dialysis may be any temperature so far as it does not deteriorate the protein synthesis activity of the wheat embryo extract-containing solution and allows dialysis. Specifically, the lowest temperature is a temperature at which the solution will not freeze, usually −10° C., and preferably −5° C. The highest temperature is a limit temperature which gives no bad influence on the solution used in dialysis, 40° C., and preferably 38° C.

In addition, if low molecular synthesis inhibitors are removed after a wheat embryo extract-containing solution is prepared, the solution needs no further stabilization component to add.

(5) Method for Decreasing Concentration of Reducing Agent in Wheat Embryo Extract-Containing Solution

The wheat embryo extract-containing solution, which contains a reducing agent at a decreased concentration, is used to execute cell-free protein synthesis, allowing production of a trigger protein which has an intramolecularly formed disulfide bond. For a method for decreasing a reducing agent in a wheat embryo extract-containing solution, there is used a method wherein a step of decreasing a reducing agent is employed anywhere in the steps for the preparation of the wheat embryo extract-containing solution. The reducing agent should be decreased to have so a concentration in the finally prepared wheat embryo extract-containing solution that the solution may be used to execute translation reaction, allowing synthesis of a trigger protein which has an intramolecularly formed disulfide bond to sustain. Dithiothreitol (hereinafter, this may be referred to as “DTT”) as a reducing agent is decreased to have a final concentration of 20 to 70 μM, and preferably 30 to 50 μM in the final solution for translation reaction prepared from a wheat embryo extract-containing solution. 2-mercaptoethanol is decreased to have a final concentration of 0.1 to 0.2 mM in the final solution for translation reaction. Glutathione/oxidized glutathione is decreased to have a final concentration of 30-50 μM/1-5 μM in the final solution for translation reaction. The specific concentration of a reducing agent is not limited to those described above and varies appropriately depending on the protein to synthesize or the kind of a cell-free protein synthesis system to use.

The method of selecting the optimal concentration range of a reducing agent is not limited in particular, and, for example, there is mentioned a selection method based on the effect of a catalyst for disulfide bond exchange reaction. Specifically, solutions for translation reaction are prepared from a wheat embryo extract-containing solution at various concentrations of a reducing agent, and then supplied with a enzyme capable of catalyzing disulfide bond exchange reaction to synthesize a trigger protein having an intramolecular disulfide bond. In addition, as a control experiment, the same protein synthesis is carried out using the same solutions for translation reaction supplied with no enzyme capable of catalyzing disulfide bond exchange reaction. Then, the soluble component of a trigger protein to synthesize is separated by a method such as centrifugation. The reaction solution, wherein this soluble component has a share of 50% (solubilization 50%) or more in the total and further has been increased by the addition of an enzyme capable of catalyzing disulfide bond exchange reaction, can be determined to be a suitable reaction solution for synthesizing a trigger protein with an intramolecular disulfide bond retained. Furthermore, within the concentration range of a reducing agent selected based on the effect of the catalyst for disulfide bond exchange reaction as above described, the concentration of the reducing agent which can synthesize the highest amount of the trigger protein can be selected as more preferable concentration range.

For specific methods for decreasing a reducing agent, there is used a method wherein a wheat embryo extract-containing solution is prepared to be free from a reducing agent, and then supplied with a reducing agent to have an above described concentration range together with necessary components for a cell-free protein synthesis system, or a method wherein a reducing agent is removed from a solution for translation reaction derived from a wheat embryo extract-containing solution to be within the concentration range described above. As a wheat embryo extract-containing solution for cell-free protein synthesis requires a high degree of reduction condition to extract, a method wherein a reducing agent is removed from the solution after extraction is easier to execute. As a method for removing reducing agent from a wheat embryo extract-containing solution, there is mentioned a method using a gelfiltration support. Specifically, for example, there is mentioned a method wherein Sephadex G-25 column is beforehand equilibrated with an appropriate buffer containing no reducing agent, and then fed with a wheat embryo extract-containing solution to pass through.

(6) Preparation of Solution for Translation Reaction

The wheat embryo extract-containing solution prepared as described above is supplied with a nuclease inhibitor, various ions, a substrate, an energy source and the like necessary for protein synthesis (hereinafter, they may be referred to as “additives for a solution for translation reaction”) and an mRNA encoding a trigger protein, which acts as a translation template, and, if desired, a stabilizer which comprising a component selected from the group consisting of inositol, trehalose, mannitol, and sucrose-epichlorohydrin copolymer to prepare a solution for translation reaction. The concentrations of components to add may be provided from a compounding ratio well known per se.

The additives for a solution for translation reaction, specifically, include amino acids acting as substrate, an energy source, various ions, a buffer, an ATP-regenerating system, a nuclease inhibitor, a tRNA, a reducing agent, polyethylene glycol, a 3′,5′-cAMP, a folate, an antimicrobial, and the like. Further, concerning each concentration, preferably, ATP is contained at 100 μM to 0.5 mM, GTP at 25 μM to 1 mM and 20 kinds of amino acids at their respective 25 μM to 5 mM. They can be selected and combined for use as appropriate according to the translation reaction system. Specifically, a wheat embryo extract, which is used for a wheat embryo extract-containing solution, is supplied with 20 mM of HEPES-KOH (pH 7.6), 100 mM of potassium acetate, 2.65 mM of magnesium acetate, 0.380 mM of spermidine (from Nacalai Tesque), respectively 0.3 mM of 20 kinds of L-amino acids, A mM of dithiothreitol, 1.2 mM of ATP (from Wako Pure Chemical Industries, Ltd.), 0.25 mM of GTP (from Wako Pure Chemical Industries, Ltd.), 16 mM of phosphocreatine (from Wako Pure Chemical Industries, Ltd.), 1000 U/ml of Rnase inhibitor (from TAKARA) and 40 μg/ml of creatine kinase (from Roche), to dissolve sufficiently, followed by adding the mRNA translation template supporting an mRNA encoding a trigger protein.

The mRNA used herein has a structure wherein the sequence encoding a trigger protein is linked in the downstream of both an appropriate sequence recognized by RNA polymerase and further a sequence having a function to activate translation. The sequence recognized by RNA polymerase includes T3 or T7 RNA polymerase promoter. Further, in a cell-free protein synthesis system, as a sequence enhancing a translation activity, there may be preferably used a sequence having a structure wherein Q sequence, Sp6 or the other is linked to the 5′-upstream of the coding sequence.

The present invention is explained in detail below with reference to Examples, but these examples are not intended to limit the scope of the present invention.

### EXAMPLE 1

Cell-Free Protein Synthesis

(1) Preparation of Wheat Embryo Extract Solution

The seeds of Chihoku wheat produced in Hokkaido or those of Chikugoizumi produced in Ehime were fed into a mill (from Fritsch: Rotor Speed Millpulverisette Type 14) at the rate of 100 g/min, and pulverized gently at a speed of 8,000 rpm. After collecting a fraction containing a germinable wheat embryo by a sieve (sieve opening from 0.7 to 1.00 mm), selection by flotation with the mixture of carbon tetrachloride and cyclohexane (volume ratio; carbon tetrachloride: cyclohexane=2.4:1) was conducted to recover a floating fraction containing a germinable wheat embryo, then organic solvent medium was dried off at room temperature, and then mixed impurities such as seed coats were removed by blowing at room temperature to obtain a crude wheat embryo fraction.

Next, using a belt type color sorter, BLM-300K (manufacturer: Anzai Manufacturing Co., Ltd., distributor: Anzai Corporation, Ltd.), a wheat embryo was sorted out from a crude wheat embryo fraction by taking advantage of differences in color as below. This color sorter is an apparatus that comprises a means for irradiating light to the crude wheat embryo fraction, a means for detecting reflected and/or transmitted beams from crude wheat embryo fraction, a means for comparing the detected value with a reference value, and a means for sorting them into classes without and within the scope of the reference value. A crude wheat embryo fraction was fed onto the beige-colored belt of the color sorter at a rate of 1000 to 5000 grains/cm², then the crude wheat embryo fraction on the belt was irradiated with the light of a fluorescent lamp and its reflected light detected. The belt was set to convey at a speed of 50 m/min. As a photosensor, a monochrome CCD line sensor (2048 pixels) was used.

First, to eliminate darker color components (seed coat etc.) than a wheat embryo, a reference value was set between the wheat embryo luminance and the seed coat luminance, and the component exceeding the reference value was sucked to eliminate. Then, to sort out an endosperm, a reference value was set between the wheat embryo luminance and the endosperm luminance, and the component exceeding the reference value was sucked to eliminate. 30 suction nozzles (1 suction nozzle per 1 cm length) were placed at the position approximately 1 cm above the conveyer belt to suck. This method was repeated to sort the wheat embryo until it has a wheat embryo purity (a weight ratio of wheat embryo contained per 1 g of random sample) of 98% or higher.

The obtained wheat embryo fraction was suspended in distilled water at 4° C., and washed with a rinsing solution in an ultrasonic cleaner until the solution got free from white turbidity. Then, it was suspended in 0.5 v % solution of Nonidet (from Nacalai Techtonics) P40, and washed with a rinsing solution in the ultrasonic cleaner until the solution got free from white turbidity to obtain wheat embryo, and then the following operations were conducted at 4° C.

An extractant (80 mM of HEPES-KOH, pH 7.8, 200 mM of potassium acetate, 10 mM of magnesium acetate, 8 mM of dithiothreitol, (each 0.6 mM of 20 kinds of L-amino acids may have been added)) of twice the volume of wet weight of the washed wheat embryo was added, and then the wheat embryo was limitedly homogenized 3 times at 5,000 to 20,000 rpm for 30 seconds using a Waring blender. This homogenate was centrifuged at 30,000×g for 30 minutes using a high-speed centrifuge to give a supernatant, which was centrifuged again in a similar condition to obtain a supernatant. This sample was subjected to long-term storage at −80° C. or below, resulting in no deterioration of the activity. The obtained supernatant was filtered with a filter having a pore size of 0.2 μm (NEW Steradisc 25: supplied by Kurabo Industries Ltd.) to sterilize by filtration and eliminate contaminating fine dusts.

Next, this filtrate was subjected to gelfiltration using Sephadex G-25 column which had been equilibrated with a solution [40 mM of HEPES-KOH (pH 7.8), and the mixture of, respectively, 100 mM of potassium acetate, 5 mM of magnesium acetate, 8 mM of dithiothreitol, each 0.3 mM of 20 kinds of L-amino acids (amino acids may be present, absent or labeled depending on the purpose of protein synthesis)] in advance. The obtained filtrate was centrifuged again at 30,000×g for 30 minutes to recover a supernatant, which was adjusted to have a concentration of 90 to 150 at A260nm (A260/A280=1.4 to 1.6). To the obtained wheat embryo extract-containing solution for protein synthesis, 20 mM of HEPES-KOH (pH 7.6), 100 mM of potassium acetate, 2.65 mM of magnesium acetate, 0.380 mM of spermidine (from Nacalai Techtonics), each 0.3 mM of 20 kinds of L-amino acids, 4 mM of dithiothreitol, 1.2 mM of ATP (from Wako Pure Chemical Industries, Ltd.), 0.25 mM of GTP (from Wako Pure Chemical Industries, Ltd.), 16 mM of phosphocreatine (from Wako Pure Chemical Industries, Ltd.), 1000 U/ml of Rnase inhibitor (from TAKARA), 400 μg/ml of creatine kinase (from Roche) were added to prepare the source of solution for translation reaction.

(2) Preparation and Translation of Transcription Template

Using a sense primer (SEQ ID NO: 1) and an anti-sense primer (SEQ ID NO: 2), the Biochain's cDNAs (derived from kidney, liver, placenta, heart, brain tissues) was used as a template to amplify by PCR, and then, a human CaMKIIδ gene (GenBANK Accession No. AF071569) which was cloned into pT7-Blue (Clonetech) vector was used to construct a transcription template by split-type PCR (Sawasaki, T. et al, PNAS, vol. 99, 14652-14657, 2002). The constructed CaMKIIδ PCR product was used as a transcription template to conduct transcription in vitro. For transcription, the transcription template made by PCR to have one tenth as much as a transcription reaction solution was supplied with 80 mM Hepes-KOH, 16 mM magnesium acetate, 2 mM spermidine (supplied by Nacalai Techtonics), 10 mM DTT, 3 mM NTP (supplied by Wako Pure Chemical Industries, Ltd.), 1 U/μl SP6 RNA polymerase and 1 U/μl Rnase Inhibitor (supplied by TAKARA) in their respective final concentrations to prepare 50 μL of the reaction solution. This reaction solution was incubated at 37° C. for three hours, and ethanol was added thereto to precipitate the mRNA. The whole pellet of the obtained mRNA was added to 50 μL ml of the extract solution of the wheat embryo extract (60 O.D.) described above in (1) to synthesize the protein.

(3) Protein Synthesis by Cell-Free Protein Synthesis System (Dialysis) in Wheat Embryo Extract Solution

CaMKIIδ protein was synthesized by dialysis. CaMKIIδ mRNA prepared in (2) described above was suspended in 50 μl of the reaction solution for protein synthesis which was supplied with the wheat embryo extract-containing solution prepared in (1) described above to have a final optical density (O.D.) (A260) of 60 [30 mM HEPES-KOH (pH7.8), 100 mM potassium acetate, 2.7 mM magnesium acetate, 0.4 mM spermidine (Nacalai Techtonics), each 0.25 mM of 20 kinds of L-amino acids, 2.5 mM dithiothreitol, 1.2 mM ATP, 0.25 mM GTP, 16 mM phosphocreatine (Wako Pure Chemical Industries, Ltd.), and 400 μg/ml creatine kinase (Roche) in their respective final concentrations]. This solution was poured in a dialysis cup MWCO 12000 (Bio Tech). In a Maruemu™ container, 700 μl of an external solution for dialysis [30 mM HEPES-KOH (pH7.8), 100 mM potassium acetate, 2.7 mM magnesium acetate, 0.4 mM spermidine (Nacalai Techtonics), each 0.25 mM of 20 kinds of L-amino acids, 2.5 mM dithiothreitol, 1.2 mM ATP, 0.25 mM GTP, 16 mM phosphocreatine (Wako Pure Chemical Industries, Ltd.) in their respective concentrations] was poured. The solution was incubated at 26° C. for a day to synthesize protein while it was often supplied with the protein's substrate and an energy source such as amino acids and ATP.

### EXAMPLE 2

(1) Assay for HeLa Cell-Extract Solution Using CaMKIIδ as Trigger

Preparation of HeLa Cell-Extract Solution

HeLa cell was cultured to confluency in a 10 cm culture dish by a conventional method. The cells were collected with a cell scraper, placed in a 50 mL centrifuge tube containing 20 mL of PBS (-) [137 mM NaCl, 8.1 mM Na₂HPO₄, 2.68 mM KCl, 1.47 mM KH₂PO₄], centrifuged (3,000 rpm, 2 min, 4° C.) to discard the supernatant, and suspended/centrifuged in 20 mL of fresh supplied PBS (-) three times to wash. The obtained cell mass was suspended again in 20 mL of PBS (-), separated into 1.5 mL tubes by every 1 mL, centrifuged (15,000 rpm, 3 min, 4° C.) to discard the supernatant and stored at −80° C. For reaction, usually, the stored 10 tubes were used to give one unit, melt, centrifuged (15,000 rpm, 5 min, 4° C.) to discard the supernatant gently, suspended again in 10 μL of a cell extract buffer [50 mM Tris-HCl (pH7.5), 1 mM EDTA, 6 mM β-mercaptoethanol], subjected to repletion of freezing in liquid nitrogen and melting at room temperature to burst cells, united to give one sample, which was then subjected to exchange of buffer and removal of intrinsic low molecular compounds using Sephadex G-25 column equilibrated with 1× Reaction Buffer [50 mM Tris hydrochloride (pH7.6), 10 mM magnesium hydrochloride, 0.5 mM dithiothreitol], and centrifuged at 15,000 rpm for 1 minute to obtain the supernatant as a cell extract solution.

(2) Phosphorylation Reaction by CaMKIIδ Using HeLa Cell Extract Solution as Substrate

To 1 μl of the CaMKIIδ protein obtained by a cell-free protein synthesis system, 3 μl of HeLa cell extract solution obtained in (1), 2 μl of 5× Activation Buffer (5 mM calcium chloride, 5 μM calmodulin derived from human brain (Alexis), 0.05 mg/ml bovine serum albumin), 1 μl of 1 mM ATP, 1 μl of 1× Reaction Buffer, 1 μl of 10% Protease inhibitor Cocktail (Sigma's code No. P8340, made by diluting 100% Protease inhibitor Cocktail with 1× Reaction Buffer by 1:10) were added and preincubated at 30° C. for 20 minutes. This reaction solution was supplied with 1 μl of [γ-³²P]ATP diluted 10 folds with 1× Reaction Buffer to make up 10 μl in total, and incubated at 30° C. for 20 minutes. The half of this reaction solution was subjected to one- or two-dimensional SDS-PAGE electrophoresis according to a conventional method, to detect the bands or spots of phosphorylated proteins by radioautography.

The results of one-dimensional SDS-PAGE electrophoresis were shown in FIG. 1. Those containing no protein obtained by a cell-free protein synthesis system (Lane 1, 2) and the dihydrofolate reductase obtained by a cell-free protein synthesis system (DHFR, Lane 3, 4) were used as control. Further, for the kinase activity of CaMKIIδ, those containing no Activation Buffer (containing calcium and calmodulin) were used as control (Lane 6, 8).

In the case with cell extract solution alone added and the case with DHFR added, significant bands were not detected. This fact shows that a wheat embryo extract solution or HeLa cell extract solution has originally a phosphorylation activity as little as background. When CaMKIIδ was added, several significant bands were observed in the presence of Activation Buffer (arrow in the figure). These bands were disappeared or weakened in the absence of Activation Buffer. It shows that CaMKIIδ expresses kinase activity depending on calcium or calmodulin. The major band of a molecule having a molecular weight of approximately 50,000 represents CaMKIIδ itself. The other bands are thought to be proteins phosphorylated by the action of CaMKIIδ in the cell extract solution.

FIG. 2 shows the results of two-dimensional electrophoresis of the reaction solutions after the similar reaction was carried out. FIG. 2A shows that of reaction conducted in the absence of Activation Buffer, FIG. 2B showed that in the presence of Activation Buffer, and FIG. 2C shows that using DHFR instead of CaMKIIδ. The spots observed in A and C show the background of phosphorylation by the activity that a wheat embryo extract solution or a HeLa cell extract solution originally has. Many spots detected in B alone are thought to be proteins phosphorylated by the action of CaMKIIδ in the cell extract solution.

In FIGS. 1 and 2, phosphorylated proteins detected in the presence of CaMKIIδ include a protein directly phosphorylated by CaMKIIδ, and another protein phosphorylated by a protein which was phosphorylated by CaMKIIδ to acquire a kinase activity, namely, a protein indirectly phosphorylated by CaMKIIδ. On the other hand, phosphorylated proteins detected in the absence of CaMKIIδ or under the condition in which CaMKIIδ's activity cannot be fully exerted (Activation Buffer-) are thought to be the other proteins dephosphorylated by the indirect action of CaMKIIδ, namely, proteins dephosphorylated by a protein which is directly or indirectly phosphorylated by CaMKIIδ to acquire a phosphatase activity.

As stated above, it was demonstrated according to the method of the present invention that CaMKIIδ induced a series of reactions on the HeLa cell extract, allowing simultaneous detection of a larger number of proteins which had been both directly and indirectly changed.

### EXAMPLE 3

(1) Identification of HeLa Cell-Derived Protein Phosphorylated by CaMKIIδ

The phosphorylated protein derived from Hela cell was identified according to a conventional method, wherein the spot of a labeled protein was cut off from the pattern of two-dimensional electrophoresis, the labeled protein was decomposed by trypsin to give peptides, their molecular weights were determined by MALDI-TOFMS, and a human protein to which five or more of the peptides coincide in molecular weight value was searched from the database. At least 20 or more kinds of CaMKIIδ-dependently phosphorylated spots were observed, two of which were identified as shown in FIG. 3. As a result, it was confirmed that spot A was eIF4B protein and spot B was stress-induced phosphoprotein 1 (STIP1) protein.

(2) Preparation of Transcription Template for Identified Gene and Translation

1 ml of TRIzol (Invitrogen) was added to 1×10⁶ HeLa cells, and RNA was extracted (final concentration 100 ng/μL) according to the protocol attached to TRIzol. The reverse transcription reaction was carried out using TaqMan reverse transcription reagents (Roche) to prepare HeLa cDNA. For cloning, the obtained HeLa cDNA described above was used as a template to amplify by PCR using sense primers (SEQ ID NOs: 3 and 5) and anti-sense primers (SEQ ID NOs: 4 and 6). Then, eIF4B gene (GenBank Accession No. AB076839) and STIP1 gene (GenBank Accession No. BC002987), which were cloned into pT7-Blue (Clonetech) vector, were used to construct a transcription template with GST gene fused to the N-terminus by the method of GST (glutathione-S-transferase) gene fusion split-typed PCR (Sawasaki, T., et al., PNAS, vol. 99, 14652-14657, 2002). Protein synthesis was carried out as in EXAMPLE 1.

(3) In Vitro Phosphorylation Assay

Reaction with CaMKIIδ was confirmed using a partially purified protein substrate which used a glutathione column. Glutathione-Sepharose 4B (Amersham) resin was washed with 10 times amount of 1× Reaction buffer three times, and a Glutathione-Sepharose 4B resin suspension was prepared in 1× Reaction buffer which had an equal volume to the column size. 20 μL of the above described resin suspension was prepared for each sample, supplied with 20 μL of reaction solutions in which GST fused protein substrates (GST-elF4B, GST-STIP1) were synthesized, and incubated (4° C., 1 h) to bind the GST fused protein substrate to the above described resin. The resin was centrifuged (800×g, 5 min) to discard the supernatant, washed once again with 200 μL of 1× Reaction buffer, centrifuged again to discard the supernatant in the same manner as above, supplied with 10 μL of CaMKIIδ exchanged in 1× Reaction buffer using SephadexG-25 column, 3 μL of 5× Activation buffer and 2 μL of 5 fold-diluted [γ-³²P] ATP, incubated (30° C., 20 min), centrifuged (800×g, 5 min) to discard the supernatant, washed three times with 200 μL of 1× Reaction buffer, supplied with 15 μL of 1×SDS-Sample buffer, and boiled for 5 minutes. 7.5 μL of the resultant was migrated (12.5% e-PAGEL, Atto Corporation) to give a gel, which was dried and subjected to autoradiography (BAS-2500, FUJI PHOTO FILM CO., LTD.) to analyze the images (FIG. 4) obtained by radiation. As a result, there was reproduced the reaction in which GST-fused eIF4B and STIP1 were phosphorylated by CaMKIIδ. Thereby, the in vitro assay also confirmed that the spots separated and identified through two-dimensional electrophoresis were eIF4B and STIP1.

(4) Semi-Intact Phosphorylation Assay

CaMKIIδ was integrated into transfection vector pcDNA3.1 (-) (Invitrogen) and the gene was introduced into HeLa cell according to a conventional method. The cells collected were supplied with 50 μL of extraction buffer and subjected to five repetitions of freeze-thawing to burst the cells. The obtained HeLa cell extract solution was exchanged in 1× Reaction buffer using Sephadex G-25 column. Then, 30 μL of buffer exchanged HeLa cell extract solution was supplied with 4.5 μL of 1 mM ATP, 3 μL of 3× Reaction buffer and 9 μL of 3× Activation buffer, preincubated (30° C., 20 min), supplied with 3 μL of [β-³²P]ATP and incubated (30° C., 20 min) to conduct phosphorylation reaction. After reaction, the whole volume was spread out by two-dimensional electrophoresis to give a gel, which was then dried and subjected to autoradiography (BAS-2500, FUJI PHOTO FILM CO., LTD.) to confirm phosphorylated spots (FIG. 5). As a result, it was demonstrated that eIF4B and STIP1 were also phosphorylated in CaMKIIδ-transfected cells. As CaMKIIδ was confirmed to express in HeLa cell, eIF4B and STIP1 are thought to be the natural substrates of CaMKIIδ.

From the results above, the in vitro screening method using the target cell extract of the present invention was demonstrated to be a method which allows identification of the natural substrate of a protein kinase in the target cell in vivo.

The present application claims priority from Japanese Patent Application Laid-open No. 2003-353949, which is incorporated herein by reference.

### BRIEF DESCRIPTION OF THE DRAWINGS

FIG. 1 shows the results of one dimensional SDS-PAGE electrophoresis.

FIG. 2A shows the results of two-dimensional electrophoresis in the absence of Activation Buffer.

FIG. 2B shows the results of two-dimensional electrophoresis in the presence of Activation Buffer.

FIG. 2C shows the results of two-dimensional electrophoresis using DHFR instead of CaMKIIδ.

FIG. 3 shows analysis charts of separation by MALDI-TOFMS of phosphorylated spots after trypsin treatment. From the peptide patterns, they were identified to be eIF4B and STIP1 respectively.

FIG. 4 shows a figure by in vitro assay. It was observed that GST-fused eIF4B and STIP1 were markedly phosphorylated by CaMKIIδ.

FIG. 5 shows a figure by semi-intact phosphorylation assay. The phosphorylation of eIF4B and STIP1 were observed in CaMKIIδ gene-transfected HeLa cell.

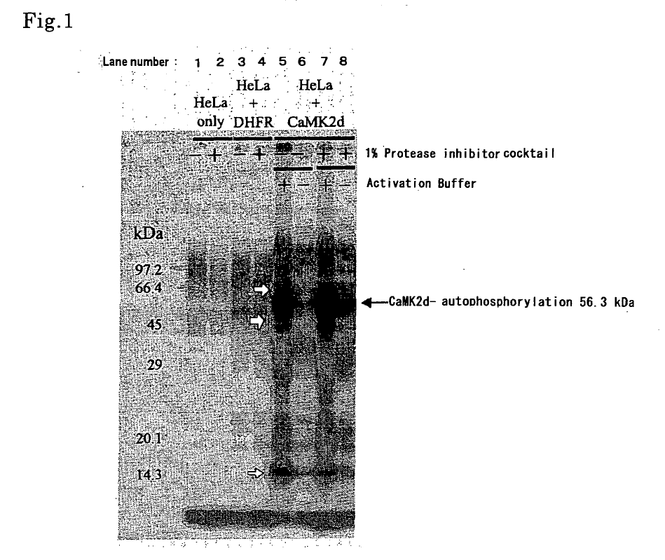

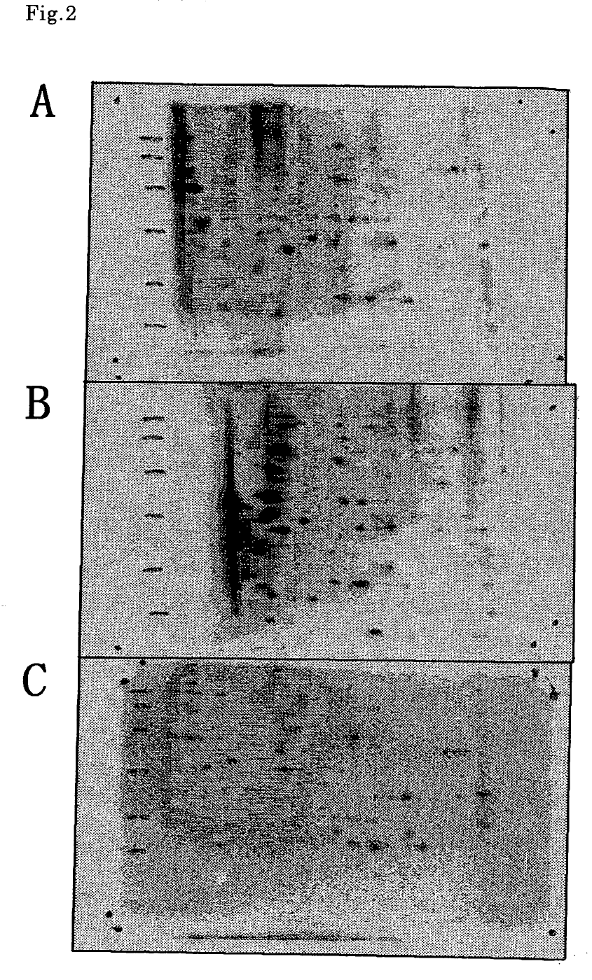

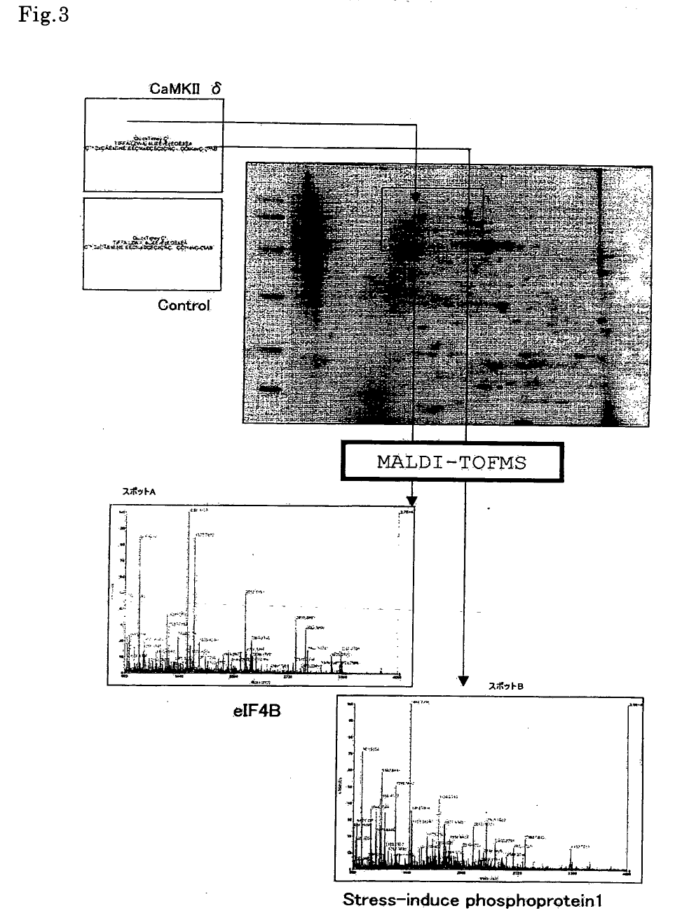

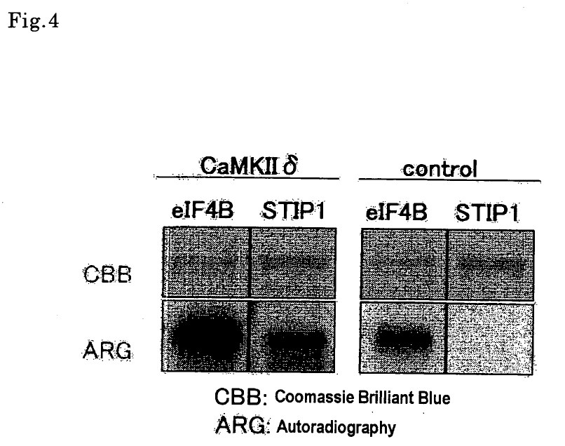

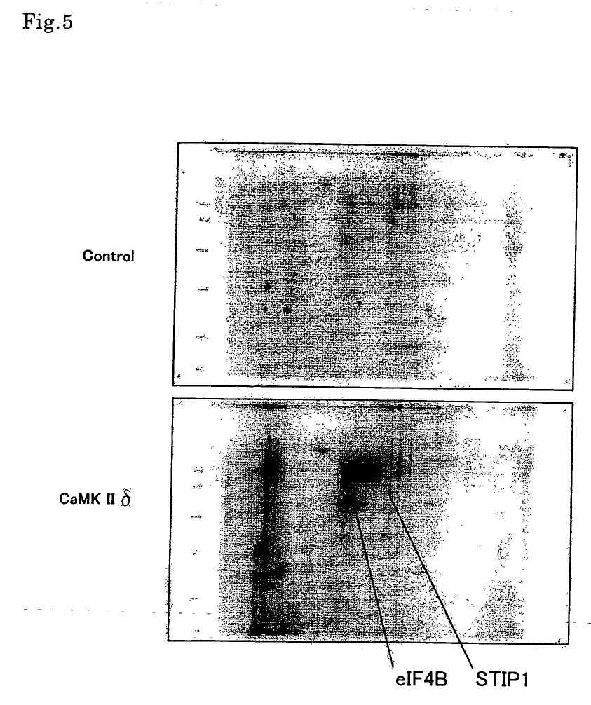

1. A method for screening an indicator substance passively produced by an action induced by a trigger protein, comprising the following steps:
1) contacting the trigger protein prepared by a cell-free protein synthesizing means with a target cell extract which contains the indicator substance that is passively produced by an action induced by the trigger protein and desired to screen, to initiate the action by the trigger protein, and
2) specifying the substance changed by the action induced by the trigger protein.

2. The screening method according to claim 1, wherein the cell-free protein synthesizing means uses a wheat embryo extract which is substantially removed from a contaminating endosperm component and a low molecular protein synthesis inhibitory substance.

3. The screening method according to claim 2, wherein the unpurified or partially purified trigger protein prepared by the cell-free protein synthesizing means initiates the action on an unspecified indicator substance.

4. The screening method according to claim 1, wherein as a marker for identifying an indicator substance changed by an action induced by a trigger protein, a particular substance capable of labeling the indicator substance of interest is introduced into the system.

5. The screening method according to claim 4, wherein the indicator substance changed by an action induced by a trigger protein is labeled with a means selected from the following:
1) radioactive substance,
2) fluorescent substance,
3) stable isotope, and
4) antibody.

6. The screening method according to claim 1, wherein the indicator substance changed by the action induced by the trigger protein is detected using a change in molecular weight as a marker.

7. The screening method according to claim 1, wherein the trigger protein is selected from the following:
1) enzyme,
2) transcription factor,
3) intranuclear receptor, and
4) cell membrane receptor.

8. The screening method according to claim 1, wherein the target cell extract is selected from the following:
1) normal cell-derived extract,
2) cancer cell-derived extract,
3) wheat embryo extract, and
4) cell-derived extract subjected to stress and/or chemical treatment.

9. A reagent kit for screening, comprising at least one reagent used in the screening method according to claim 1.

10. An indicator substance which is passively produced by an action induced by a trigger protein identified by the screening method according to claim 1.

11. A method for screening a substance affecting the action of a trigger protein on a target cell extract, comprising: using the indicator substance specified in claim 10 as a control, contacting the trigger protein with the target cell extract in the presence or absence of the candidate substance, and comparing changes in the specified indicator substance.

12. The screening method according to claim 2, wherein as a marker for identifying an indicator substance changed by an action induced by a trigger protein, a particular substance capable of labeling the indicator substance of interest is introduced into the system.

13. The screening method according to claim 3, wherein as a marker for identifying an indicator substance changed by an action induced by a trigger protein, a particular substance capable of labeling the indicator substance of interest is introduced into the system.

14. The screening method according to claim 2, wherein the indicator substance changed by the action induced by the trigger protein is detected using a change in molecular weight as a marker.

15. The screening method according to claim 3, wherein the indicator substance changed by the action induced by the trigger protein is detected using a change in molecular weight as a marker.

16. The screening method according to claim 2, wherein the trigger protein is selected from the following:
1) enzyme,
2) transcription factor,
3) intranuclear receptor, and
4) cell-membrane receptor.

17. The screening method according to claim 3, wherein the trigger protein is selected from the following:
1) enzyme,
2) transcription factor,
3) intranuclear receptor, and
4) cell membrane receptor.

18. The screening method according to claim 2, wherein the target cell extract is selected from the following:
1) normal cell-derived extract,
2) cancer cell-derived extract,
3) wheat embryo extract, and
4) cell-derived extract subjected to stress and/or chemical treatment.

19. The screening method according to claim 3, wherein the target cell extract is selected from the following:
1) normal cell-derived extract,
2) cancer cell-derived extract,
3) wheat embryo extract, and
4) cell-derived extract subjected to stress and/or chemical treatment.
